(19) United States
(12) Patent Application Publication
(10) Pub. No.: US 2014/0242016 A1
(43) Pub. Date: Aug. 28, 2014

(54) NON-AQUEOUS SOLID STABILIZED EMULSIONS

(71) Applicant: Stiefel Laboratories, Inc., Wilmington, DE (US)

(72) Inventors: Bernard Paul BINKS, Walkington (GB); Russell Elliott, Research Triangle Park, NC (US); Paul David Ian Fletcher, Hull (GB); Andrew James Johnson, Cottingham (GB); Michael Andrew Thompson, Cottingham (GB)

(73) Assignee: Stiefel Laboratories, Inc., Wilmington, DE (US)

(21) Appl. No.: 14/186,063

(22) Filed: Feb. 21, 2014

Related U.S. Application Data
(60) Provisional application No. 61/767,880, filed on Feb. 22, 2013.

(51) Int. Cl. A61K 8/19; A61Q 15/00

(52) U.S. Cl. CPC A61K 8/19; A61Q 15/00
USPC 424/68; 514/770

(57) ABSTRACT

The present invention relates to non-aqueous emulsions stabilised by silica particles and to processes for making them. The invention further comprises:
a particle stabilized oil-in-polar (o/polar o/p) emulsion comprising:
a) a dispersed oil phase,
b) a continuous polar phase substantially free of water,
c) a particulate solid which is a silica particle dispersant possessing surface silanol groups (SiOH) sufficient to stabilize the emulsion, and where the emulsion is substantially free of emulsifiers, surfactants; and
   wherein the oil phase (a) is dispersed as discontinuous droplets in the polar phase (b); the silica particle dispersant (c) is absorbed on the surface of the oil phase (a); and the silica particle dispersant (c) is partially wetted by the polar phase (b).

### FIELD OF THE INVENTION

The present invention relates to non-aqueous emulsions stabilised by silica particles and to processes for making them.

### BACKGROUND OF THE INVENTION

The skin forms a protective barrier that keeps harmful toxins out and essential fluids in. Types of irritants that irritate the epidermal barrier include detergents and surfactants, which can irritate the epidermal barrier by reducing skin thickness and by diminishing the skin's barrier. Repeated use of surfactants makes the skin drier and more prone to irritation by other factors. Therefore, dermatological formulations that do not include surfactants would be highly desirable.

When an oil in water (o/w) emulsion is prepared using two poorly miscible components, e.g. water and oil, a suitable surfactant is usually used to enhance the emulsification and to make the thus formed emulsion stable. Both o/w and water-in-oil (w/o) emulsions have been widely used in various fields such as food, agrochemical, pharmaceutical, cosmetic, paint and oil industries. This is due to the unique properties of emulsions distinct from homogeneous solutions, and the opportunities available from having nano- and/or micro-scale droplets dispersed in a continuous phase.

Preparation of emulsions typically requires utilization of surface-active agents (surfactants) and/or amphiphilic polymers, and energy input (e.g., homogenizers and ultrasonicators). Emulsions have been investigated in terms of molecular characteristics of surfactants and/or amphiphilic polymers, and their resulting interfacial properties.

Several groups have been working on o/w surfactant-free o/w emulsion preparation (e.g., an oil droplet dispersion in water in the absence of any stabilizing agents) using a number of different techniques and methodologies.

Solid colloidal particles are widely used in many industries such as food, cosmetic, paper and paint. In the case of emulsion systems, where solid nanoparticles act as effective stabilizing agents for emulsions, these are categorized as surfactant-free emulsions (particle-stabilized surfactant-free emulsions). See B. P. Binks, Curr. Opin. Colloid Interface Sci. 2002, 7, 21-41; E. Vignati, R. Piazza, T P. Lockhart, Langmuir 2003, 19, 6650-6656; S. Stiller, H. Gers-Barlag, M. Lergenmueller, F. Pflücker, J. Schulz, K P. Wittern, R. Daniels, Colloids Surf A 2004, 232, 261-267. Particle-stabilized emulsions exhibit unique phase inversion as a function of the oil:water content ratio and pH. Colloidal stability of surfactant-free o/w emulsions can be enhanced with the addition of long-chain hydrocarbons or hydrophobic polymers into short-chain hydrocarbons to prevent Ostwald ripening. Ultrafine particles of inorganic and organic substances are usually prepared in the presence of a surfactant, and the removal of the adsorbed surfactant molecule from the surface of the ultrafine particle is difficult.

A surfactant stabilized emulsion and two surfactant free aqueous (o/w) based emulsions stabilized by silica particles (e.g., a Pickering emulsion) were evaluated using skin absorption assays for use with lipophilic drugs. (See Frelichowska, et al., Int. J. of Pharmaceutics 371 (2009) 56-63).

Other emulsifier free o/w formulations include those cited in U.S. Pat. No. 6,295,339 Binks et al.; US2011/0178207 Gottschalk-Gaudig et al.; and U.S. Pat. No. 7,722,891 Barthel et al. Schonrock et al. U.S. Pat. No. 5,804,167 discloses emulsifier free cosmetic or dermatological formulations for w/o preparations. Gers-Barlag et al. U.S. Pat. No. 6,709,662 also discloses emulsifier free w/o and o/w preparations. Gers-Barlag et al. U.S. Pat. No. 5,725,844 discloses waterproof emulsifier free w/o and o/w preparations. Collin et al., U.S. Pat. No. 5,643,555 also discloses surfactant free w/o emulsions for use in the cosmetic field.

The effect of adding polar media to an aqueous dispersion of nanoparticles and paraffin liquid-water emulsions stabilized by these same particles is described in various papers, such as S. R. Raghavan et al., Langmuir, 2000, 16, 7920, and H. Bartel, Colloids and Surfaces A: Physiochemical and Engineering Aspects, 1995, 101, 217. Pickering emulsions and their use in cosmetic preparations with carbonyl iron particles is described in S. Melle, et al., Langmuir 2005, 21, 2158-2162.

There still exists a need for the development of surfactant-free emulsions in non-aqueous colloidal containing systems which systems can provide significant opportunities for pharmaceutical, veterinary and cosmetic agents not suitable for formulating in aqueous systems.

### BRIEF DESCRIPTION OF THE DRAWINGS

FIG. 1 shows the conductivity and type of emulsions prepared from 50 vol. % paraffin liquid and 50 vol. % PEG300 containing 1 wt. % silica particles as a function of particle hydrophobicity.

FIGS. 2 A and B show the appearance and type of emulsions prepared from 50 vol. % paraffin liquid and 50 vol. % PEG300 containing 1 wt. % silica particles as a function of particle hydrophobicity after 1 day (upper) and 1 week (lower). The % SiOH on the silica is illustrated for each vial.

FIGS. 3 A, B and C show the optical micrographs of dilute paraffin liquid/PEG 300 emulsions (φₒ=0.5) stabilised by 1 wt. % silica particles of varied wettability, viewed immediately after preparation. The % SiOH on the silica is shown for each figure, FIG. 14A=14% SiOH, with a scale bar of 50 μm; FIG. 14B=23% SiOH, with a scale bar of 50 μm; FIG. 14C=51% SiOH, with a scale bar of 50 μm.

FIG. 4 shows the conductivity and type of emulsions prepared from 50 vol. % Miglyol 812 and 50 vol. % propane-1,2-diol containing 1 wt. % silica particles as a function of particle hydrophobicity.

FIGS. 5 A and B show the appearance and type of emulsions prepared from 50 vol. % Miglyol 812 and 50 vol. % propane-1,2-diol containing 1 wt. % silica particles as a function of particle hydrophobicity after 1 day (upper) and 1 week (lower). The % SiOH on the silica is illustrated.

FIGS. 6 A-D show the optical micrographs of dilute Miglyol 812/propane-1,2-diol emulsions (Φₒ=0.5) stabilised by 1 wt. % silica particles of varied wettability, viewed immediately after preparation. The % SiOH on the silica in FIG. 6A is 14% SiOH, with a scale bar of 500 μm; FIG. 6B=23% SiOH, with a scale bar of 100 μm; FIG. 6C=37% SiOH, with a scale bar of 100 μm and in FIG. 6D=51% SiOH, with a scale bar of 100 μm.

### SUMMARY OF THE INVENTION

The present invention relates to a particle stabilized oil-in-polar (o/p) emulsion comprising:
(a) a dispersed oil phase,
(b) a continuous polar phase substantially free of water,
(c) a silica particle dispersant possessing surface silanol groups (SiOH) sufficient to stabilize the emulsion, and where the emulsion is substantially free of emulsifiers surfactants, and stabilizing polymers; and
wherein the oil phase (a) is dispersed as discontinuous droplets in the polar phase (b); the silica particle dispersant (c) is absorbed on the surface of the oil phase (a); and the silica particle dispersant (c) is partially wetted by the polar phase (b).

The present invention also relates to a particle stabilized polar-in-oil (p/o) emulsion which comprises:
a) a continuous oil phase,
b) a dispersed polar phase substantially free of water,
c) a silica particle dispersant possessing surface silanol groups (SiOH) sufficient to stabilize the emulsion, and where the emulsion is substantially free of emulsifiers, surfactants, and stabilizing polymers; and
wherein the polar phase (b) is dispersed as discontinuous droplets in the oil phase (a); the silica particle dispersant (c) is absorbed on the surface of the polar phase (b); and the silica particle dispersant is partially wetted by the oil phase (a).

In both of the above embodiments the emulsions may further comprise an electrolytic component soluble in the polar phase.

In another embodiment, the oil phase and polar phase are of equal volume, and may range from a volume ratio of 1:99 to 75:25.

In another embodiment, the polar phase comprises a diol, substantially free from water. In one embodiment the diol is selected from ethane-1,2-diol, propane-1,3-diol, propane-1,2-diol, butane-1,4-diol, butane-1,3-diol, butane-1,2-diol, or polyethylene glycol.

In another embodiment there is provided for a process for preparation of an oil-in-polar (o/p) emulsion comprising:
a) a dispersed oil phase;
b) a continuous polar phase substantially free of water;
c) a silica particle dispersant possessing surface silanol groups (SiOH) sufficient to stabilize the emulsion, and where the emulsion is substantially free of emulsifiers, surfactants, and stabilizing polymers;
d) adding the silica particle dispersant (c) as a powder on top of the most dense liquid phase of (a) or (b);
e) adding the least dense phase of (a) or (b) on top of (d); and
f) mixing or homogenizing (e) to produce an emulsion; and
wherein the oil phase (a) is dispersed as discontinuous droplets in the polar phase (b); the silica particle dispersant (c) is absorbed on the surface of the oil phase (a); and the silica particle dispersant is partially wetted by the polar phase (b).

In another embodiment there is provided for a process for preparation of a polar-in-oil (p/o) emulsion comprising:
a) a continuous oil phase;
b) a dispersed polar phase substantially free of water;
c) a silica particle dispersant possessing surface silanol groups (SiOH) sufficient to stabilize the emulsion, and where the emulsion is substantially free of emulsifiers, surfactants, and stabilizing polymers;
d) adding the silica particle dispersant (c) as a powder on top of the most dense liquid phase of (a) or (b);
e) adding the least dense phase of (a) or (b) on top of (d); and
f) mixing or homogenizing (e) to produce an emulsion; and
wherein the polar phase (b) is dispersed as discontinuous droplets in the oil phase (a); the silica particle dispersant (c) is absorbed on the surface of the polar phase (b); and the silica particle dispersant is partially wetted by the oil phase (a).

### DETAILED DESCRIPTION OF THE INVENTION

Stable non-aqueous emulsions without the use of emulsifiers and surfactants provide a highly desirable base for use in the pharmaceuticals and cosmetic industry. Surface active agents are generally low molecular substances which contain one or more polar groups and also contain one or more non-polar groups. These surface active agents are often classified as cationic, anionic or non-ionic. They accumulate at the interfaces of these formulations, such as in liquid-liquid, liquid-solid or liquid-gas interfaces and reduce the interfacial surface tension or energy.

These agents can also cover the surface of a substrate, thus affecting the wetting properties of that surface. This can adversely affect the properties of the formulation, or in many instances be a desired effect. An ordinary emulsion contains dispersed drops which can become unstable over time. It is desirable to obtain an emulsion which is not only non-aqueous, but stable over long periods of time. Many pharmaceutical and cosmetic agents degrade, or are not soluble in, aqueous solutions or emulsions and therefore need to be formulated in alternative dispersions.

The emulsions according to the invention are substantially free of conventional liquid and solid organic surface-active substances such as non-ionic, cationic and anionic emulsifiers.

The emulsions according to the invention can be used for cosmetic and pharmaceutical applications, and can include pharmacologically active drug substances. The emulsions according to the invention are substantially stable to separation, i.e. substantially stable to creaming or sedimentation of the disperse phase and thus provide the opportunity for a longer shelf life. This may be of particular importance for a dermatological or cosmetic product.

As used herein, the term “substantially stable to separation” means that the volume of the phase depleted in the dispersion is less than 10% of the total volume. In one embodiment the volume of the phase depleted is less than 5% of the total volume. In another embodiment the volume of the phase depleted is less than 1% of the total volume.

The present invention provides for a formulation and a process of forming a particle stabilized o/p emulsion comprising,

a) a dispersed oil phase,

b) a continuous polar phase substantially free of water,

c) a silica particle dispersant possessing surface silanol groups (SiOH) sufficient to stabilize the emulsion, and where the emulsion is substantially free of emulsifiers, surfactants; and

wherein the oil phase (a) is dispersed as discontinuous droplets in the polar phase (b); the silica particle dispersant (c) is absorbed on the surface of the oil phase (a); and the silica particle dispersant (c) is partially wetted by the polar phase (b).

In an alternative embodiment of the invention, there is a formulation and a process for forming a particle stabilized polar-in-oil (w/o) emulsion which comprises:
a) a continuous oil phase,
b) a dispersed polar phase substantially free of water,
c) a silica particle dispersant possessing surface silanol groups (SiOH) sufficient to stabilize the emulsion, and where the emulsion is substantially free of emulsifiers, surfactants, and stabilizing polymers; and

wherein the polar phase (b) is dispersed as discontinuous droplets in the oil phase (a); the silica particle dispersant (c) is absorbed on the surface of the polar phase (b); and the silica particle dispersant is partially wetted by the oil phase (a).

Improved stability may be indicated by improved storage life (“shelf-life”), before the dispersion separates into its components. As a more conventional emulsion containing a surface active agent may have a relatively long storage time, it is difficult to compare a dispersion with a surface active agent with a dispersion, such as described herein without one.

Without oil, aerated mixtures of aqueous propylene glycol and particles yield stable dispersions, aqueous foams, climbing particle films and liquid marbles, depending on the glycol content present in the formulation and the resulting particle hydrophobicity. The particles behave as if they are more hydrophilic in the presence of glycol. In the presence of oil, these particle-stabilised emulsions will invert from a w/o emulsion to an o/w emulsion upon increasing either the hydrophilicity of the particles, or the glycol content in the system. Using calculated contact angles at the oil-polar phase interface, reasonable agreement is found between measured and calculated phase inversion conditions.

The presence of glycol in water promotes particles to behave as if they were more hydrophilic. It has been found that calculations of their contact angle at the air-aqueous propylene glycol surface are in agreement with this. In the presence of an oil, particle-stabilised emulsions invert from a w/o to an o/w emulsion upon increasing either the inherent hydrophilicity of the particles, or the glycol content in the aqueous phase. Stable multiple emulsions occur around phase inversion in systems of low glycol content.

Immiscible mixtures of oil and water may be made kinetically stable by addition of an emulsifier to form emulsions in which drops of one of the liquids become dispersed in the continuous phase of the other liquid. (See Colloidal Particles at Liquid Interfaces, eds. B. P. Binks and T. S. Horozov, Cambridge University Press, Cambridge, 2006, p. 1). Stable emulsions occur in a wide range of industries including the food, personal care, cosmetic, oil field, chemical and pharmaceutical sectors. Certain pharmaceutical emulsions incorporating paraffin oil may be administered either topically to the skin or injected directly, can contain high concentrations of polar glycol (or diol) species such as propane-1,2-diol (propylene glycol).

Propylene glycol has many other applications industrially including use as a humectant, a moisturizer, a carrier in fragrance oils and as a non-toxic antifreeze agent. Although some information exists on the stabilization of emulsions of oil and non-aqueous polar liquids, little is known on emulsions containing water-diol mixtures as the polar phase. (See D. Hamill et al., J. Pharm. Sci., 1966, 55, 1268, and 1274; A. Imhof et al., J. Colloid Interface Sci., 1997, 192, 368; and M. Klapper, et al., Acc. Chem. Res., 2008, 41, 1190).

The ability of particles to stabilise emulsions of oil and water depends, inter alia, on their wettability at the interface. (B. P. Binks, Curr. Opin. Colloid Interface Sci., 2002, 7, 21). This wettability is quantified through the three-phase contact angle θ (measured through the aqueous phase). For equal volumes of oil and water, hydrophilic particles of θ<90° stabilise o/w emulsions, whereas hydrophobic particles of θ>90° stabilise w/o emulsions. The change in free energy accompanying desorption of a spherical particle from the oil-water interface to either bulk phase is given by (See A. F. Koretsky et al., Izv. Sib. Otd. Akad. Nauk USSR, 1971, 2, 139; and B. P. Binks et al., Langmuir, 2000, 16, 8622):

ΔE=πr²γ_{ow}(1±cos θ)² (1)

in which r is the particle radius, γ_{ow} is the bare oil-water interfacial tension and the plus sign refers to desorption into oil whilst the minus sign refers to that into water. At fixed particle size and interfacial tension, ΔE is maximum at θ=90° since this situation corresponds to the maximum area of interface obliterated by placing the particle at it. Systems in which ΔE is large (several hundred kT where k is the Boltzmann constant and T is the absolute temperature) exhibit contact angles of intermediate values (not close to 0 or) 180° and produce the most stable emulsions to coalescence. Conversely, particles of very low or very high θ are not well held at the interface (ΔE very low) and give rise to emulsions of low coalescence stability. (See B. P. Binks et al., Langmuir, 2000, 16, 8622; N. W. Yan, et al, Colloids Surf A, 2001, 193, 97; and S. Stiller, et al, Colloids Surf A, 2004, 232, 261).

Stabilisation of emulsions composed of polar liquids, other than water, has previously required exotic surfactants or polymers. Thus, the present invention has determined that emulsions of polar liquids could be stabilized using silica particles in which the hydrophobicity (akin to the surfactant hydrophile-lipophile balance number) (M. Klapper et al., Acc. Chem. Res., 2008, 41, 1190) can be systematically varied. Suitable particles, such as those used herein have been previously found to be a stabilizer for many kinds of aqueous o/w type of emulsions with an optimum particle hydrophobicity being required depending on the oil type. (B. P. Binks et al., Phys. Chem. Chem. Phys., 2000, 2, 2959).

Miscible mixtures of water and propane-1,2-diol both in the absence and presence of an oil for a range of silica particles of different inherent hydrophobicity were investigated. Rationalization of that data was reviewed in terms of the influence of propane-1,2-diol on the contact angles of the particles at the air-polar phase or oil-polar phase interfaces.

### DEFINITIONS

As used herein, the term “about” indicates a deviation of +/−10% of the given value, preferably +/−5% and most preferably +/−2% of the numeric values, when applicable.

As used herein, the term “non-aqueous” and “water-free” solvent system means that no water is specifically added to a formulation as described herein. The terms “water-free” and “non-aqueous” do not exclude the presence of trace amounts of water present in the formulation, such as less than 5%, preferably less than 3% starting materials, and more preferably less than 1% w/w.

The term “substantially free” means that the volume of the object that the formulation is free from, e.g. surfactant, water, etc. in that phase or final formulation is less than 10% of the volume or total volume. In one embodiment the volume is less than 5% of volume or total volume. In another embodiment the volume is less than 1% of the volume of the phase or the total volume, as appropriate.

As used herein, a substance is considered to be lipophilic when it has an affinity for fat and has high lipid solubility. Lipophilicity is thus a physicochemical property which describes the partitioning equilibrium of solute molecules between water and an immiscible organic solvent, favoring the latter. Lipophilicity is generally expressed by the partition coefficient, Log P, between water and a water-immiscible solvent. One solvent commonly used in drug discovery and development is 1-octanol. Log P refers to the logarithm of the Partition Coefficient, P, which is defined as the ratio of concentration of neutral species in octanol divided the concentration of neutral species in water.

As used herein the terms “active agent”, “drug moiety” or “drug” are all used interchangeably. The terms “mold” and “mould” are also used interchangeably herein.

As used herein, “vitamin analogue” includes compounds that are derived from a particular vitamin, and thus are similar in structure and have similar chemical and physiological properties.

As used herein, the terms “stabilizer,” or “preservative” includes an agent that prevents the oxidation or degradation of other compounds, or the growth of unwanted agents.

### Drug Substance

Drug substances or pharmaceutically or cosmetically acceptable agents (as can be used interchangeably herein) can be highly potent and/or toxic compounds with small or narrow therapeutic windows. The drug or drugs will be present in an amount needed to generate a pharmacological effect in the targeted tissue, such as by application to the skin. According to an embodiment of the invention, said drug is present in an amount of about 0.01 to about 30% by weight based on the total weight of the composition.

In one embodiment the drug substance is a lipophilic drug. In one embodiment the drug substance is suitable for nutritional or cosmetic use.

In another embodiment the drug substance is an oil-soluble UV filter substance, a deodorant or antiperspirant, an antioxidant, an insect repellent, a vitamin, or an antimicrobial agent.

In one embodiment the drug substance is one or more cosmetically or pharmaceutically acceptable oil-soluble UV filter substances.

The oil-soluble UV filter substances according to the invention can be chosen from substances which absorb UV radiation chiefly in the UVB range, or a mixture thereof, and the total amount of filter substances being, for example, 0.1% by weight to 30% by weight. In one embodiment the amount is from about 0.1 to 15% w/w. In another embodiment, the amount is from about 0.5 to 10% by weight. In another embodiment, the amount is from about 0.5 to 8.0% by weight, based on the total weight of the formulation.

Suitable oil-soluble UVB filters are, for example:
3-benzylidenecamphor derivatives, such as 3-(4-methylbenzylidene) camphor and 3-benzylidenecamphor;
4-aminobenzoic acid derivatives, such as 2-ethylhexyl 4-(dimethylamino)-benzoate and amyl 4-(dimethylamino)benzoate;
esters of cinnamic acid, such as 2-ethylhexyl 4-methoxycinnamate and isopentyl 4-methoxycinnamate;
esters of salicylic acid, such as 2-ethylhexyl salicylate, 4-isopropylbenzyl salicylate and homomethyl salicylate;
derivatives of benzophenone, such as 2-hydroxy-4-methoxybenzophenone, 2-hydroxy-4-methoxy-4′-methylbenzophenone and 2,2′-dihydroxy-4-methoxybenzophenone;
esters of benzalmalonic acid, such as di-(2-ethylhexyl) 4-methoxybenzalmalonate;
triazine derivatives, such as 2,4,6-trianilino-(p-carbo-2′-ethyl-1′-hexyloxy)-1,3,5-triazine and tris(2-ethylhexyl) 4,4′,4″-(1,3,5-triazine-2,4,6-triyltriimino)trisbenzoate,
benzotriazole derivatives, such as 2,2′-methylenebis(6-(2H-benzotriazol-2-yl)-4-(1,1,3,3,-tetramethylbutyl)phenol) and UV filters bonded to polymers.

Where appropriate, it may be advantageous to incorporate water soluble UV filter substances into the polar solvent phase of formulations according to the invention, alone or in combination with the oil-soluble UV filers. Advantageous water-soluble UVB filters are, for example:

salts of 2-phenylbenzimidazole-5-sulphonic acid, such as its sodium, potassium or its triethanolammonium salt, and the sulphonic acid itself;

sulphonic acid derivatives of benzophenones, such as 2-hydroxy-4-methoxybenzophenone-5-sulphonic acid and its salts, e.g. benzophenone-3.

sulphonic acid derivatives of 3-benzylidenecamphor, such as, 4-(2-oxo-3-bornylidenemethyl)-benzenesulphonic acid, and 2-methyl-5-(2-oxo-3-bornylidenemethyl) sulphonic acid and its salts.

The list of UV-B filters mentioned which can be used in the Pickering emulsions according to the invention is of course not intended to be limiting.

It can also be advantageous to use UV-A filters in the emulsions according to the invention which have been customarily present in other cosmetic preparations. These substances are suitably derivatives of dibenzoylmethane, such as 1-(4′-tert-butylphenyl)-3-(4′-methoxyphenyl)propane-1,3-dione and 1-phenyl-3-(4′-isopropylphenyl)propane-1,3-dione.

Other advantageous UV-A filter substances are phenylene-1,4-bis(2-benzimidazyl)-3,3′-5,5′-tetrasulphonic acid and its salts, such as the corresponding sodium, potassium or triethanolammonium salts; or the bis-sodium salt of phenylene-1,4-bis(2-benzimidazyl)-3,3′-5,5′-tetrasulphonic acid and 1,4-di(2-oxo-10-sulfo-3-bornylidenemethyl)benzene and salts thereof (such as the corresponding 10-sulfato compounds, and the corresponding sodium, potassium or triethanolammonium salt), also referred to as benzene-1,4-di(2-oxo-3-bornylidenemethyl-10-sulphonic acid).

Preparations which comprise UV-A filters are also provided for by this invention, alone or in combination with a UV-B filter. The amounts which can be used are similar to those used for the UV-B combination and are well known in the art.

In particular useful sunscreen agents for incorporation into an emulsion herein are: Diethylamino Hydroxybenzoyl Hexyl Benzoate (DHHB) (Uvinul MC80); Bemotrizinol (BEMT) (Tinosorb S); Iscotrizinol (DBT) (Uvasorb HEB); Ethylhexyl Triazone (Uvinul T150); Bisoctrizole (MBBT) (Tinosorb M); butyl methoxydibenzoylmethane (Avobenzone); Bisdisulizole Disodium (Neo-Heliopan AP); diethylhexyl syringylidene malonate (Oxynex ST); Octocrylene; Ethylhexyl Salicylate (Octisalate); Isoamyl p-Methyoxy-cinnamate (Neo-Heliopan E1000); homosalate; Drometrizole Trisiloxane (Mexoryl XL); and/or 2-ethylhexyl 4-(dimethylamino)benozate, 2-ethyl hexyl dimethyl PABA (Padimate O), alone or in combination or mixtures thereof.

Antioxidants are also suitable for incorporation herein as an active substance. Suitable antioxidants include but are not limited to, vitamin C and derivatives (e.g. ascorbyl palmitate, Mg ascorbyl phosphate, ascorbyl acetate), the tocopherols (vitamin E) and derivatives (e.g. vitamin E acetate), folic acid, phytic acid (inositolhexaphosphoric acid, also fytic acid), the various ubiquinones (mitoquinones, coenzyme Q), bile extract, cis- and/or trans-urocanic acid (4-imidazolylacrylic acid), D,L-carnosine, D-carnosine, L-carnosine and derivatives thereof (e.g. anserine), flavones or flavonoids, cystins (3,3′-dithiobis(2-aminopropionic acid)), cystsine (2-amino-3-mercaptopropionic acid), propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, .gamma.-linoleyl, cholesteryl and glyceryl esters thereof) and the salts thereof, carotenes (α-carotene, β-carotene and lycopene), tyrosine (2-amino-3-(4-hydroxyphenyl)-propionic acid), α-liponic acid (1,2-dithiolane-3-pentanoic acid) and derivatives (e.g. dihydrolipoic acid), glutathione (gamma-L-glutamyl-L-cysteineglycine) and glutathione esters, furalglucitol (sorbitylfurfural), mannitol and zinc and zinc derivatives, such as zinc oxide and zinc salts (for example ZnSO₄); amino acids (e.g. glycine, histidine, tyrosine, tryptophan) and derivatives thereof, imidazoles, (e.g. urocanic acid) and derivatives thereof, chlorogenic acid and derivatives thereof, aurothioglucose, dilauryl thiodipropionate, distearyl thiodipropionate, thiodipropionic acid and derivatives thereof (esters, ethers, peptides, lipids, nucleotides, nucleosides and salts) and sulphoximine compounds (e.g. buthionine sulphoximines, homocysteine sulphoximine, buthionine sulphones, penta-, hexa-, hepta-thionine sulphoximine) in very low tolerated doses (e.g. pmol to μmol/kg), and also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin A and derivatives (vitamin A palmitate) and coniferyl benzoate of benzoin resin, rutinic acid and derivatives thereof, α-glycosylrutin, ferulic acid, furfurylideneglucitol, carnosine, butylated hydroxytoluene, butylated hydroxyanisole, nordihydroguaiac acid, nordihydroguaiaretic acid, trihydroxybutyrophenone, uric acid and derivatives thereof, mannose and derivatives thereof, selenium and its derivatives (e.g. selenomethionine), stilbenes and their derivatives (e.g. stilbene oxide, trans-stilbene oxide), and the derivatives (salts, esters, ethers, sugars, nucleotides, nucleosides, peptides and lipids) of said active substances which are suitable according to the invention.

Those antioxidants which are oil-soluble antioxidants are suitably advantageous for use in the present invention.

The amount of the above mentioned antioxidants (one or more compounds) in the preparations according to the invention is preferably from 0.001 to 30% by weight, particularly preferably from 0.05-20% by weight, in particular 1-10% by weight, based on the total weight of the preparation.

If vitamin E and/or derivatives thereof are used as the antioxidant or antioxidants, their respective concentrations are advantageously chosen from the range of 0.001-10% by weight, based on the total weight of the formulation.

If vitamin A or vitamin A derivatives or carotenes or derivatives thereof are used as the antioxidant or antioxidants, their respective concentrations are advantageously chosen from the range of 0.001-10% by weight, based on the total weight of the formulation.

The total amount of antioxidants can advantageously be 0.1% by weight to 30% by weight, preferably 0.5 to 10% by weight, in particular 1 to 6% by weight, based on the total weight of the formulation.

Cosmetic deodorants are used to control body odor which arises when fresh perspiration, which is in itself odorless, is decomposed by microorganisms. Customary cosmetic deodorants are based on various modes of action. In antiperspirants, astringents, mainly aluminum salts, such as aluminum hydroxychloride (aluminum chlorohydrate), reduce the formation of perspiration.

The use of antimicrobial substances in cosmetic deodorants can also reduce the bacterial flora of the skin. In an ideal situation, only the microorganisms which cause the odor should be effectively reduced. The flow of perspiration itself is not influenced as a result, and in ideal circumstances, only microbial decomposition of perspiration is stopped temporarily.

The combination of astringents and antimicrobial active substances in one and the same composition is also common

Deodorants or antiperspirants may also be included as an active agent in the emulsions of the present invention. Antibacterial agents are also suitable to be incorporated into the novel emulsions herein. Suitable substances include but are not limited to, 2,4,4′-trichloro-2′-hydroxy diphenyl ether (Irgasan), 1,6-di(4-chlorophenylbiguanido)hexane (chlorhexidine), 3,4,4′-trichlorocarbanilide, quaternary ammonium compounds, oil of cloves, mint oil, thyme oil, triethyl citrate, farnesol (3,7,11-trimethyl-2,6,10-dodecatrien-1-o1).

The list of specified active ingredients and active ingredient combinations is of course not intended to be limiting.

The amount of antiperspirant active ingredients or deodorants (one or more compounds) in the preparations is preferably from 0.01 to 30% by weight, particularly preferably from 0.1 to 20% by weight, in particular 1-10% by weight, based on the total weight of the preparation.

“Pharmaceutically acceptable agents” includes, but is not limited to, drugs, proteins, peptides, nucleic acids, nutritional agents, as described herein. This term includes therapeutic active agents, bioactive agents, active agents, therapeutic agents, therapeutic proteins, diagnostic agents, or drug(s) as defined herein, and follows the guidelines from the European Union Guide to Good Manufacturing Practice (GMP). Such substances are intended to furnish pharmacological activity or other direct effect in the diagnosis, cure, mitigation, treatment, or prevention of a disease or to affect the structure and function of the body. The substance may also include a diagnostic agent, such as an imaging agent and/or a radioactive labelled compound, which may be used to diagnose disease or for generating information relating to the structure and function of the gastrointestinal regions. The substances use may be in a mammal, or may be in a human. The pharmaceutical compositions described herein may optionally comprise one or more pharmaceutically acceptable active agents, bioactive agents, active agents, therapeutic agents, therapeutic proteins, diagnostic agents, or drug(s) or ingredients distributed within. Water solubility of an active agent is defined by the United States Pharmacoepia. Therefore, active agents which meet the criteria of very soluble, freely soluble, soluble and sparingly soluble as defined therein are encompassed this invention.

Suitable drug substances can be selected from a variety of known classes of drugs including, but not limited to, analgesics, anti-inflammatory agents, anthelmintics, anti-arrhythmic agents, antibiotics (including penicillins), anticoagulants, antidepressants, antidiabetic agents, antiepileptics, antihistamines, antihypertensive agents, antimuscarinic agents, antimycobactefial agents, antineoplastic agents, immunosuppressants, antithyroid agents, antiviral agents, anxiolytic sedatives (hypnotics and neuroleptics), astringents, beta-adrenoceptor blocking agents, blood products and substitutes, cardiac inotropic agents, corticosteroids, cough suppressants (expectorants and mucolytics), diagnostic agents, diuretics, dopaminergics (antiparkinsonian agents), haemostatics, immunological agents, lipid regulating agents, muscle relaxants, parasympathomimetics, parathyroid calcitonin and biphosphonates, prostaglandins, radiopharmaceuticals, sex hormones (including steroids), anti-allergic agents, stimulants and anorexics, sympathomimetics, thyroid agents, phosphodiesterase inhibitors, neurokinin inhibitors, CSBP/RK/p38 inhibitors, antipsychotics, vasodilators and xanthines.

Preferred drug substances include those intended for topical and oral administration. In one embodiment the drug substance is for use topically. A description of these classes of drugs and a listing of species within each class can be found in Martindale, The Extra Pharmacopoeia, Twenty-ninth Edition, The Pharmaceutical Press, London, 1989, the disclosure of which is hereby incorporated herein by reference. These drug substances are commercially available and/or can be prepared by techniques known in the art.

In one embodiment the water-insoluble or oil soluble drug substance may include an analgesic such as capsaicin or piroxicam, an antifungal such as clotrimazole or miconazole nitrate, an antibacterial such as nitrofurazone or gramicidin, an anaesthetic such as benzocaine or lidocaine, an antiviral such as acyclovir or penciclovir, an antipruritic such as crotamiton or phenol, an antihistamine such as chlorpheniramine or triprolidine, a xanthine such as caffeine, a sex hormone such as oestradiol or testosterone, or an anti-inflammatory agent, such as capsaicin, or a corticosteroid may be used.

One or more suitable corticosteroids may be selected, hydrocortisone, hydrocortisone acetate, fluticasone propionate, alclometasone dipropionate, fluclorolone acetonide, amcinonide, fluocinolone acetonide, beclamethasone dipropionate, fluocinonide, betamethasone benzoate, fluocortin butyl, betamethasone valerate, betamethasone dipropionate, fluocortolone preparations, fluprednidene acetate, budesonide, flurandrenolone, clobetasol propionate, halcinonide, clobetasone butyrate, desonide, desoxymethasone, hydrocortisone butyrate, diflorasone diacetate, methylprednisolone acetate, diflucortolone valerate, mometasone furoate, flumethasone pivalate, triamcinolone acetonide, and mixtures thereof.

Combinations of active ingredients are also within the scope of the present invention.

Vitamins and analogues thereof are also suitable active ingredients of the present invention. As used herein, “vitamins” include vitamins such as vitamin A, B₁, B₂, B₃, B₅, B₆, B₇, B₉, B₁₂, C, D₁, D₂, D₃, D₄, and K.

As used herein, “vitamin analogue” includes compounds that are derived from a particular vitamin, and thus are similar in structure and have similar chemical and physiological properties. Vitamin analogues useful in the present invention include naturally occurring and synthetic analogues. Vitamin analogues of the present invention include, but are not limited to, calcidiol, calcitriol, calcipotriene, paricalcitol, 22-oxacalcitriol, dihydrotachysterol, calciferol, and those listed in U.S. Pat. No. 6,787,529. Vitamin A analogues useful in the present invention include, but are not limited to, acitretin, retinaldehyde, retinoic acid, dehydroretinol, fenretinide, hydroxyretroretinol, didehydroretinoic acid, carotenes, tretinoin and its isomers. One of skill in the art will appreciate that other vitamin analogues are useful in the present invention.

The drug substances which are suitable for inclusion in the polar phase, may first be dissolved in at least one of the polar soluble solvents, such as propylene glycol. Other solvents having miscibility with both polar and non-polar substances can be used including for example, diols such as ethylene glycol, butylene glycol and other polyols. Other solvents having miscibility with both polar and non-polar substances can also be used included; polyols, for example PEG 200, PEG 300, PEG 400 and PEG 800; and ethers, for example, ethylene glycol monoethyl ether and diethylene glycol monoethyl ether; and esters, for example ethyl acetate and propylene carbonate; and heterocyclic compounds, for example n-methylpyrrolidone. For particular agents (e.g., tretinoin), alcohols are useful, such as ethanol, n-propanol, isopropanol, n-butanol and t-butanol.

Exemplary nutritional agents for use herein also include coenzymes, fruit extracts, plant extracts, and mixtures thereof.

In one embodiment the drug substance is a lipophilic drug.

In another embodiment the lipophilic drug substance is an immunomodulator or immune response modifier. In one embodiment when the drug is an immunomodulator, it is a toll like receptor (TLR7) ligand. Examples of existing TLR7 agent include, but are not limited to, imiquimod or/and resiquimod. According to another embodiment of the invention, the immunomodulator can be a corticosteroid.

If desired, the cosmetic or dermatological formulations according to the invention can furthermore comprise cosmetic auxiliaries such as are usually used in such formulations, for example amino acids, preservatives, bactericides, substances having a deodorizing action, dyestuffs, pigments having a coloring action, thickening agents, softening substances, moisturizing and/or moisture-retaining substances, fats, oils, waxes or other customary constituents of a cosmetic formulation. In general, if it is intended to incorporate more oil than that amount described above, a lipophilic gelling agent may be added, which makes it possible to increase the quantity of oil while maintaining good emulsion stability and while avoiding a greasy appearance when this emulsion is applied to the skin. Lipophilic gelling agents which may be used include modified clays such as bentones, metal salts of fatty acids, such as aluminum stearate, and hydrophobic silica and glycol stearate esters such as the acetylated glycol stearate ester sold by Guardian under the name of Unitwix.

### Non-Aqueous Polar Solvent

The polar solvent, or mixture of polar solvents, suitable for use herein are taken from the group of compounds such as aromatic alcohols such as benzyl alcohol, cyclic alcohols such as cyclohexanol, diacetone alcohol, ethylene glycol monomethyl ether, diethylene glycol monomethyl ether, ethylene glycol monoethyl ether, diethylene glycol monoethyl ether, oleyl alcohol, short chain mono-aliphatic alcohols having up to 8 carbon atoms, such as ethanol, propanol and isopropanol, di-, or tri-polyhydric alcohols having from about 2 to 12 carbon atoms, such as ethane-1,2-diol, propane-1,3-diol, propane-1,2-diol (also known as propylene glycol), butane-1,2-diol, butane-1,3-diol, butane-1,4-diol, pentane-1,5-diol, 1,2-hexanediol or polyethylene glycol; tri-hydric or polyhydric alcohols, include but are not limited to glycerin or glycerol (also known as 1,2,3-propanetriol), butanetriol, or 1,2,6-hexanetriol; glycols, such as polyethylene glycol, ethylene glycol, ethyl glycol, butylene glycol, diethylene glycol, dipropylene glycol, ethyl hexanediol, ethylene glycol, hexylene glycol, pentylene glycol, propylene glycol, propylene glycol monolaurate, tetraethylene glycol, triethylene glycol, tripropylene glycol, polyethylene glycol and polypropylene glycol; and alkylated sulfoxides, such as dimethylsulfoxide, and mixtures thereof, and all of which are substantially free of water.

Suitable glycols may be in monomeric or polymeric form and include polyethylene and polypropylene glycols such as PEG 4-200, which are polyethylene glycols having from 4 to 200 repeating ethylene oxide units; as well as C₁₋₆ alkylene glycols such as propylene glycol, butylene glycol, pentylene glycol, hexanediol, and the like.

Examples of polyethylene glycols (PEG's) are of the formula: HOCH₂(CH₂OCH₂)ₙ OH, wherein n represents the average number of oxyethylene groups. Polyethylene glycols are commercially available such as those from Dow Chemical, and are designated by a number such as 200, 300, 400, 600, 2000, which represents the approximate average molecular weight of the resulting polymer. Polyethylene glycols 200, 300, 400 and 600 are clear viscous liquids at room temperature.

In one embodiment the nonaqueous polar solvent is a C₁₋₆, preferably C₂₋₄ alkylene glycols, most particularly ethylene, propylene, or butylene glycol, or a mixture thereof.

In one embodiment the nonaqueous polar solvent is glycerin or a mixture thereof.

In one embodiment the nonaqueous polar solvent is ethanol or isopropanol, or a mixture thereof.

In one embodiment the polar solvent is propylene glycol.

In one embodiment the polar solvent is present in an amount of 1 to 80 weight %, based on the total weight of the composition.

In one embodiment the polar phase solvent is propane-1,2-diol, present in an amount of about 1% to about 50% of total volume of the two phases for both an o/p and a p/o emulsion.

It is recognized that in all instance the polar phase components must be either a liquid or soluble in one or more of the other polar phase components to remain liquid for use in the present invention.

### Oils or Lipids

The oil or lipid phase is a nonpolar substance which is largely immiscible with water or the polar solvent.

Suitable oils of lipids can consist of hydrocarbons, be it aliphatic or aromatic, although for pharmaceutical and cosmetic purposes it is unlikely that benzene, toluene or xylene would be used. Aliphatic hydrocarbons such as pentanes, hexanes e.g., n-hexane, cyclohexanes, heptanes, octane, e.g., n-octane and isooctanes, nonanes, decanes, undecanes and dodecanes may play some role in the pharmaceutical and cosmetic industries but all are suitable for non-human usage and function as an embodiment of this invention. Other oil or lipids suitable for use include alkenes and poly-alkenes, esters, ethers, polyethers, ketones, and long-chain alcohols, e.g. n-octanol, and organosilicon compounds such as silicones, e.g. linear or cyclic polydialkylsiloxanes, polydimethylsiloxanes having 0-10% by weight of methylsiloxy and/or trimethylsiloxy units in addition to 90-100% by weight of dimethylsiloxy units, or any mixtures thereof. These lists of the oil phase substances are exemplary, and not limiting.

Other oils and lipids useful in the present invention include but are not limited to fats, natural or synthetic fat substances such as fatty alcohols, fatty acids, esters of fatty acids, and esters of glycerin, fatty alcohols, waxes, sterols, unsaponifiables, siloxanes, silanes, lanolin, hydrocarbons, glyceryl esters, essential oils, vegetable oils, fruit oils, mineral oils, animal oils, edible oils, natural oils, including triglycerides such as caprylic or capric acids; alkyl benzoates; silicon oils, phospholipids, or processed hydrocarbons, and/or fluorinated oils, and light oils such as isohexadecane.

It is recognized that in all instances the oil phase components must be either a liquid or soluble in one or more of the other oil phase components to remain liquid for use in the present invention.

Formulations according to the invention the oil phase may comprise from about 0.5-75% by weight of the total composition. In one embodiment the oil phase may comprise from about 0.5 to 55% by weight of the total composition. In one embodiment the oil phase may comprise from about 0.5 to 35% by weight of the total composition.

The oils may be volatile or nonvolatile, and are in the form of a pourable liquid at room temperature. The term “volatile” means that the oil has a measurable vapor pressure, or a vapor pressure of at least about 2 mm of mercury at 20° C. The term “nonvolatile” means that the oil has a vapor pressure of less than about 2 mm of mercury at 20° C.

Suitable volatile oils generally have a viscosity ranging from about 0.5 to 5 centistokes at 25° C. and include linear silicones, cyclic silicones, paraffinic hydrocarbons, or mixtures thereof.

Linear and cyclic volatile silicones are available from various commercial sources including Dow Corning Corporation and General Electric. The Dow Corning volatile silicones are sold under the tradenames Dow Corning 244, 245, 344, and 200 fluids. These fluids comprise octamethylcyclotetrasiloxane, decamethylcyclopentasiloxane, dodecamethylcyclohexasiloxane and the like. Also suitable are linear volatile silicones such as hexamethyldisiloxane (viscosity 0.65 centistokes (abbreviated cst)), octamethyltrisiloxane (1.0 cst), decamethyltetrasiloxane (1.5 cst), dodecamethylpentasiloxane (2 cst) and mixtures thereof.

Various types of fluorinated oils may also be suitable for use in the compositions including but not limited to fluorinated silicones, fluorinated esters, or perfluoropolyethers. In one embodiment for use herein are the fluorosilicones such as trimethylsilyl endcapped fluorosilicone oil, polytrifluoropropylmethylsiloxanes, and similar silicones such as those disclosed in U.S. Pat. No. 5,118,496. Perfluoropolyethers include those disclosed in U.S. Pat. Nos. 5,183,589, 4,803,067, 5,183,588 and commercially available from Montefluos under the trademark Fomblin.

Volatile Paraffinic Hydrocarbons include various straight or branched chain paraffinic hydrocarbons having 5-20 carbon atoms, suitably 8 to 16 carbon atoms. Such hydrocarbons include pentane, hexane, heptane, decane, dodecane, tetradecane, tridecane, and C₈₋₂₀ isoparaffins.

Exemplary esters of glycerin include, but are not limited to, caprylic/capric triglyceride, capryl glucoside, cetearyl glucoside, coco-glucoside, decyl glucoside and lauryl glucoside.

More specifically glyceryl esters of fatty acids or triglycerides are suitable for use in the compositions. Both vegetable and animal sources may be used. Examples of such oils include castor oil, lanolin oil, C₁₀₋₁₈ triglycerides, caprylic/capric/triglycerides, sweet almond oil, apricot kernel oil, sesame oil, camelina sativa oil, tamanu seed oil, coconut oil, corn oil, cottonseed oil, linseed oil, ink oil, olive oil, palm oil, illipe butter, rapeseed oil, soybean oil, grapeseed oil, sunflower seed oil, walnut oil, and the like.

Also suitable are synthetic or semi-synthetic glyceryl esters, such as fatty acid mono-, di- and triglycerides which are natural fats or oils that have been modified, for example, mono-, di- or triesters of polyols such as glycerin. In one example, a fatty (C₁₂₋₂₂) carboxylic acid is reacted with one or more repeating glyceryl groups such as glyceryl stearate, diglyceryl diiosostearate, polyglyceryl-3 isostearate, polyglyceryl-4 isostearate, polyglyceryl-6 ricinoleate, glyceryl dioleate, glyceryl diisotearate, glyceryl tetraisostearate, glyceryl trioctanoate, diglyceryl distearate, glyceryl linoleate, glyceryl myristate, glyceryl isostearate, PEG castor oils, PEG glyceryl oleates, PEG glyceryl stearates, PEG glyceryl tallowates, and so on.

Monoesters are esters formed by the reaction of a monocarboxylic acid having the formula R—COOH, wherein R is a straight or branched chain saturated or unsaturated alkyl having 2 to 45 carbon atoms, or phenyl; and an alcohol having the formula R—OH wherein R is a straight or branched chain saturated or unsaturated alkyl having 2-30 carbon atoms, or phenyl. Both the alcohol and the acid may be substituted with one or more hydroxyl groups. Either one or both of the acid or alcohol may be a “fatty” acid or alcohol, and may have from about 6 to 30 carbon atoms, more preferably 12, 14, 16, 18, or 22 carbon atoms in straight or branched chain, saturated or unsaturated form. Examples of monoester oils that may be used in the compositions of the invention include hexyl laurate, butyl isostearate, hexadecyl isostearate, cetyl palmitate, isostearyl neopentanoate, stearyl heptanoate, isostearyl isononanoate, stearyl lactate, stearyl octanoate, stearyl stearate, isononyl isononanoate, and so on.

Diesters are the reaction product of a dicarboxylic acid and an aliphatic or aromatic alcohol or an aliphatic or aromatic alcohol having at least two substituted hydroxyl groups and a monocarboxylic acid. The dicarboxylic acid may contain from 2 to 30 carbon atoms, and may be in the straight or branched chain, saturated or unsaturated form. The dicarboxylic acid may be substituted with one or more hydroxyl groups. The aliphatic or aromatic alcohol may also contain 2 to 30 carbon atoms, and may be in the straight or branched chain, saturated, or unsaturated form. In one embodiment, one or more of the acid or alcohol is a fatty acid or alcohol, i.e. contains 12-22 carbon atoms. The dicarboxylic acid may also be an alpha hydroxy acid. The ester may be in the dimer or trimer form. Examples of diester oils that may be used in the compositions of the invention include diisotearyl malate, neopentyl glycol dioctanoate, dibutyl sebacate, dicetearyl dimer dilinoleate, dicetyl adipate, diisocetyl adipate, diisononyl adipate, diisostearyl dimer dilinoleate, diisostearyl fumarate, diisostearyl malate, dioctyl malate, and so on.

Suitable triesters comprise the reaction product of a tricarboxylic acid and an aliphatic or aromatic alcohol or alternatively the reaction product of an aliphatic or aromatic alcohol having three or more substituted hydroxyl groups with a monocarboxylic acid. As with the mono- and diesters mentioned above, the acid and alcohol contain 2 to 30 carbon atoms, and may be saturated or unsaturated, straight or branched chain, and may be substituted with one or more hydroxyl groups. In one embodiment, one or more of the acids or alcohols is a fatty acid or alcohol containing 12 to 22 carbon atoms. Examples of triesters include esters of arachidonic, citric, or behenic acids, such as triarachidin, tributyl citrate, triisostearyl citrate, tri C₁₂₋₁₃ alkyl citrate, tricaprylin, tricaprylyl citrate, tridecyl behenate, trioctyldodecyl citrate, tridecyl behenate; or tridecyl cocoate, tridecyl isononanoate, and so on.

Most fatty alcohols in nature are generally waxes, e.g. esters of fatty acids and fatty alcohols.

Exemplary fatty alcohols include, but are not limited to, caprylic alcohol, decyl alcohol, lauryl alcohol, myristyl alcohol, behenyl alcohol, lanolin alcohol, arachidyl alcohol, oleyl alcohol, palm alcohol, isocetyl alcohol, cetyl alcohol and stearyl alcohol, or a combination or mixture thereof.

Exemplary fatty acids include, but are not limited to, isoarachidic acid, linoleic acid, linolenic acid, myristic acid, palmitic acid, ricinoleic acid, sterculic acid, aleurtic acid and arachidic acid.

Exemplary waxes include, but are not limited to, beeswax, carnauba wax, dimethicone PEG-1 beeswax, dimethiconol beeswax, lanolin wax, microcrystalline wax, white wax, candelilla wax, paraffin wax, emulsifying wax, PEG-8 beeswax, shellac wax and synthetic beeswax.

Exemplary sterols include, but are not limited to, Brassica Campestris sterols, C10-C30 cholesterol/lanosterol esters, canola sterols, cholesterol, glycine soja sterols, PEG-20 phytosterol and phytosterols.

Exemplary siloxanes and silanes include, but are not limited to, dimethicone, phenyl dimethicone, cyclopentasiloxane, cyclotetrasiloxane, dimethyl siloxane and dimethicone cross polymer.

Exemplary hydrocarbons oils include, but are not limited to, include paraffinic hydrocarbons and olefins such as those having greater than about 20 carbon atoms, e.g. C₂₄₋₂₈ olefins, C₃₀₋₄₅ olefins, C₂₀₋₄₀ isoparaffins, hydrogenated polyisobutene, polyisobutene, polydecene, hydrogenated polydecene, mineral oil, pentahydrosqualene, squalene, squalane, cyclohexane, dodecane, hexane, isobutane, isopentane, petrolatum, paraffin, and pentane and mixtures thereof.

Exemplary essential oils include, but are not limited to, primrose oil, rose oil, eucalyptus oil, borage oil, bergamot oil, chamomile oil, citronella oil, lavender oil, peppermint oil, pine oil, spearmint oil, tea tree oil and wintergreen oil.

Exemplary vegetable oils include, but are not limited to, almond oil, aniseed oil, apricot oil, canola oil, castor oil, coconut oil, corn oil, fish oil, avocado oil, cottonseed oil, olive oil, palm kernel oil, peanut oil, safflower oil, soybean oil and vegetable oil.

Exemplary mineral oils include, but are not limited to, mineral oil and light mineral oil.

Exemplary edible oils include, but are not limited to, cinnamon oil, clove oil, lemon oil and peppermint oil.

In an embodiment, the oil phase comprises a mixture of one or more oils.

In one embodiment the mixture is of paraffin oil or mineral oil, and a triglyeride. In one embodiment the triglyceride is caprylic/capric triglyceride.

### Particulate Materials

All particulate solids are useful, in particular finely divided particulate solids which are insoluble in both the polar phase and the oil phase, and are thus present in the emulsion as particles. Suitable particulate solids for use herein include the include phyllosilicates, e.g. clays, such as laponites, bentonites, and montmorillonites; solid polymers, e.g. polystyrene; inorganic carbonates such as calcium carbonates, including natural calcium carbonates, preferably ground and classified, and precipitated synthetic calcium carbonates; sulfates such as barium sulfate, e.g. natural, ground and classified barium sulfates or else precipitated barium sulfate; nitrides, e.g. boron nitride and silicon nitride; carbides, e.g. boron carbide and silicon carbide; and metal oxides, e.g. titanium dioxides, aluminum dioxides, zirconium dioxides and silicon dioxides. Among the silicon dioxides are included e.g. kieselguhr or diatomaceous earths which are natural and ground or classified by processes such as dispersion and sedimentation, and also synthetic silicon dioxides, e.g. silicon dioxides precipitated by wet-chemical methods or prepared pyrogenically in a flame. Preference is given to pyrogenic silicon dioxides which are prepared in a flame process by reacting silicon compounds which can be evaporated up to 300 C°, preferably up to 150 C°., e.g. SiCl₄, CH₃SiCl₃, HSiCl₃, HCH₃SiCl₂, mixtures thereof, including mixtures contaminated with other Si compounds and/or hydrocarbons up to 20% by weight, preferably up to 10% by weight, preferably in a hydrogen/oxygen flame, the latter preferably in a substantially stoichiometric mixture, “substantially” referring to less than a 20% deviation from stoichiometry.

It is possible to use any desired mixtures of the abovementioned particles. Preference is given to mixtures of hydrophilic, polar solvent-wettable and hydrophobic, polar solvent-unwettable particles. In one embodiment there is a mixing ratio of hydrophilic to hydrophobic particles of from 1:4 to 4:1. In another embodiment the ratio is from 1:2 to 2:1.

In one embodiment, the emulsions include particulate solids which comprise at least one metal oxide. In another embodiment the particulate solids comprise at least silicon dioxide. In another embodiment the particulate solids comprise hydrophobic silicon dioxide or at least partially silylated silicon dioxide. In another embodiment the particulate solids comprise a mixture of hydrophilic and hydrophobic silicon dioxide. In yet another embodiment the particulate solids comprise pyrogenically prepared silicon dioxide.

For the particles according to the invention, while all typical material densities are possible, suitably, the particle size is less than 1 micrometer. In one embodiment the particle size is less than 100 nm. In another embodiment the particle size is less than 60 nm, based on the average diameter of the primary particles. In another the primary particle is less than 30 nm. In another embodiment the primary particle is from about 5 nm to 60 nm.

In one embodiment preference is given to using pyrogenic silicon dioxide. The silicon dioxide preferably has an average primary particle size less than 100 nm. In one embodiment the average primary particle size if from about 5 to about 60 nm. In another embodiment the average primary particle size is about 30 nm. These primary particles generally do not exist in isolated form within the silicon dioxide, but are constituents of larger aggregates and agglomerates. The silicon dioxide in one embodiment has a specific surface area of from 25 to 500 m²/g (measured according to the BET method in accordance with DIN 66131 and 66132).

For the emulsions herein all particle shapes are possible, such as spherical, discoid, rod-like, branched, e.g. fractal, with fractal dimensions for the mass Dₘ of 1<Dₘ<3. In one embodiment, the particles are spherical. In another embodiment the particles have a branched and/or fractal structure.

The silicon dioxide most likely has aggregates (definition in accordance with DIN 53206) in the range of diameters from 50 to 1000 nm. In one embodiment the stable aggregate of the silica primary particle dispersants is about 100 to about 500 nm in diameter. The agglomerates (definition in accordance with DIN 53206) are constructed from aggregates, which have sizes from 1 to 500 μm depending on the external shear stress (e.g. measurement conditions).

The emulsion of any of the preceding claims, wherein the residual density of surface silanol groups is about 14% to about 100% of the silica particle's surface area (as defined by original silanol content).

In one embodiment, the residual density of surface silanol groups is about 14%, 23%, 27%, 42%, 51%, 61%, 71%, 88% or 100%.

In one embodiment, for a polar-in-oil emulsion, the residual density of surface silanol groups is about 30% or less. In another embodiment, the residual density of surface silanol groups is about 26% or less.

In one embodiment, for an oil in polar emulsion, the residual density of surface silanol groups is about 30% or greater. In another embodiment, the residual density of surface silanol groups is about 27% or greater.

In the present invention the silica dispersant suitably has a calculated contact angle θ of θ<90° for o/w emulsions. In one embodiment the 50-130 degree angle will demonstrate optimal interfacial properties.

In the present invention the silica dispersant suitably has a calculated contact angle θ of θ>90° for w/o emulsions.

The present invention uses commercially available solid silica particles that are chemically modified to achieve the desired hydrophobicity. These particles initially contain on their surface up to 100% (SiOH) silanol groups. For purposes herein, the modification of the silanol groups changes the hydrophobicity/hydrophilicity of the particle. Without modification, the commercially available solid silica particles are hydrophilic. In all instances, the silica particles and modified silica particles remain solid in the emulsions and not solubilized in the oil or polar solvent phases. Not only do they remain as a particulate, they localize at the interface of the two phases and remain there throughout. The energy required to move the particles from the interface is so large, that they remain in place and give a stable emulsion to coalescence. This is in contrast to solubilized surfactants, and in particular solubilized alkyl dimethicone copolyols, that are molecular stabilizers. These molecular stabilizers are in dynamic equilibrium (e.g. surfactants come on and off the interface of the two phases) and therefore can be prone to destabilization.

As used herein, “sufficient to stabilize” implies that the particles have the appropriate contact angle in the given emulsion so that coalescence is reduced, hence stability is increased. Contact angles around 90 degrees (50-130 degrees) need to be produced. This is achieved by preparing a series of emulsions where a parameter is altered that will change the particle contact angle, one example for instance in this case is changing the particle hydrophobicity and/or the nature of the polar solvent and oil phase. Sufficient stabilization will occur when the emulsion transitions from polar-in-oil to oil-in-polar or vice versa, as, at or around that transition the particle is at an approximate contact angle of 90 degrees and therefore the energy required to remove the particle from the interface is at its highest, and therefore sufficient to stabilize the two immiscible phases. For clarity, particles can stabilize emulsions over a contact angle range of approx 50-130 degrees, which again is understood from measuring the emulsion transition from polar-in-oil to oil-in-polar or vice versa as discussed above.

The preferred starting silica, from which the silica used in the emulsions according to the invention and partly wettable with water or polar solvent, can be prepared in any desired manner known per se, such as, for example, in a flame reaction from halogen-silicon compounds, for example from silicon tetrachloride, or halogen-organosilicon compounds, such as methylchlorosilanes, such as methyltrichlorosilane, or hydrogenchlorosilanes, such as hydrogentrichlorosilane, or other hydrogenmethylchlorosilanes, such as hydrogenmethyldichlorosilane, or alkylchlorosilanes, also as a mixture with hydrocarbons, or any desired sprayable and, preferably, volatilizable mixtures of organosilicon compounds, as mentioned, and hydrocarbons, it being possible for the flame to be a hydrogen-oxygen flame or a carbon monoxide-oxygen flame. The preparation of the silica can be effected alternatively with or without further addition of water, for example in the purification step; preferably, no water is added.

It is possible to use silicon dioxides as noted above prepared at elevated temperature (>1000 C°). Suitably the silicon dioxides are prepared pyrogenically. It is also possible to use hydrophilic silicon dioxides which come freshly prepared direct from the burner, which have been stored temporarily, or have already been packaged in a standard commercial manner. It is also possible to use hydrophobized silicon dioxides, e.g. standard commercial products. It is also possible to use uncompacted silicon dioxides with bulk densities of less than 60 g/l, and also compacted silicon dioxides with bulk densities greater than 60 g/l. It is also possible to use mixtures of different silicon dioxides, for example mixtures of silicon dioxides of varying BET surface area, or mixtures of silicon dioxides with a different degree of hydrophobization or silylation.

The process for hydrophobization or partial hydrophobing, and in particular the silylation or partial silylation, of particles, in particular of metal oxides, and especially of silicon dioxide, can be carried out by conventional techniques known to the skilled artisan. Mixtures of different silicas can be used as starting silicas, for example mixtures of silicas of different BET surface area.

Analysis of the coverage of particles, in particular metal oxides, and especially silicon dioxide, with hydrophobicizing agents or silylating agents, can be carried out via the determination of the carbon content from elemental analysis, via IR methods such as DRIFT and ATIR, via adsorption methods which are based on the BET methodology, as described in S. Brunnauer, et al., J. Am. Chem. Soc. (JACS), 1938, Vol. 60, p. 309, and as further disclosed in Barthel et al., U.S. Pat. No. 7,722,891 which is incorporated by reference herein. The determination of the acidic OH groups on metal oxide surfaces, especially the residual silicon dioxide silanol groups on the surface of silicon dioxides, can, for example, take place by acid-base titrations following the process in accordance with G. W. Sears, Anal. Chem., 28 (1956) 510.

Suitably, partly hydrophobized, and preferably partly silylated, silica sinter aggregates are used as silica sinter aggregates for the preparation of the emulsions according to the invention. Here, partly silylated means that neither is the total silica surface unsilylated nor is the total silica surface silylated.

The coverage with silylating agent can be determined, for example, by means of elemental analysis, such as, for example, via the carbon content, or by determination of the residual content of reactive surface silanol groups of the silica sinter aggregates.

Partial silylation furthermore means that the content of non-silylated surface silanol groups on the silica surface is from not more than 95% to not less than 5%, more preferably from 90 to 10%, in particular from 85 to 25%, of the silanol groups of the starting silica.

The pyrogenic silica is arranged at the oil-water interface and is partly silylated in a manner suitably that the content of non-silylated surface silanol groups on the silica surface is from not more than 95% to not less than 5% of the starting silica, equivalent to from 1.7 to 0.1 SiOH groups per nm² of silica surface, the dispersion component of the surface energy gamma-s-D is from 30 to 80 mJ/m² and the specific BET surface area has a value of from 30 to 500 m²/g.

For the silylation of silicas, organosilicon compounds may be used such as those described in Gottschalk-Gaudig et al., US 2011/0178207 and incorporated by reference herein.

The emulsions according to the invention contain sinter aggregates of suitable pyrogenic silicas, where the sinter aggregates are arranged at the oil-polar interface. The sinter aggregates used according to the invention are sinter aggregates partly wettable with polar and oil phase.

### Additives

Examples of preservatives useful in the compositions of the present invention include, but are not limited to, an antioxidant, sodium nitrate, sodium nitrite, sulfites, (sulfur dioxide, sodium bisulfate, potassium hydrogen sulfate, and the like), disodium EDTA, formaldehyde, glutaraldehyde, diatomaceous earth, ethanol, dimethyl dicarbonate, methylchloroisothiazolinone, beta-carotene, selenium, coenzyme Q10 (ubiquinone), lutein, tocotrienols, soy isoflavones, S-adenosylmethionine, glutathione, taurine, N-acetylcysteine, Vitamin E (alpha-tocopherol), Vitamin E derivatives such as tocopherol acetate and tocopherol palmitate, Vitamin C and its derivatives, alpha-lipoic acid, 1-carnitine, phenoxyethanol, butylated hydroxytoluene and sodium benzoate. One of skill in the art will appreciate that other preservatives are useful in the present invention. When a preservative is present, it is typically present in an amount of from about 0.1% to about 5% by weight.

Other preservative/stabilizers useful in the present invention include complexing agents such as EDTA disodium, dihydrate. When a complexing agent is present, it is present in an amount of from about 0.001% to about 1%. One of skill in the art will appreciate that other complexing agents, and amounts, are useful in the present invention.

The method of preparation and characterisation of water/glycol based Pickering emulsions has been reported before, (“Understanding and optimisation of non-conventional emulsions”, Ph.D. Thesis, Michael Thompson, University of Hull, July 2012), however the method of preparation of non-aqueous/waterless Pickering emulsions will now be outlined.

### Experimental and Materials

Propane-1,2-diol (propylene glycol) (Dow Corning, 98% purity, racemic mixture) and polyethylene glycol (PEG300) (Sigma Aldrich, molecular weight 285-315 g mol-i) were used as received. Paraffin liquid oil (Total, grade 783LP), was columned over neutral alumina to remove polar impurities. The Paraffin liquid oil is a mixture of heavier alkanes (C₁₂-C₂₀) and has a density of 0.86 g cm₋₃ at 25° C. Miglyol 812 (Sasol, Batch 110711) was also columned over neutral alumina to remove polar impurities.

Fumed silica particles with different hydrophobicities were provided by Wacker-Chemie (Germany). The hydrophilic silica particles, possessing surface silanol groups (SiOH) and with a surface area of 200 m² g⁻¹, from which the others are derived are produced by hydrolysis of silicon tetrachloride in an oxygen-hydrogen flame at high temperature. In the flame process, molecules of SiO₂ collide and coalesce to give smooth and approximately spherical primary particles of 10-30 nm in diameter. These primary particles collide and may fuse at lower temperatures to form stable aggregates of 100-500 nm in diameter. Hydrophobization is achieved by reacting hydrophilic silica with dichlorodimethylsilane (DCDMS) in the presence of molar amounts of water, followed by drying at 300° C. for 1 hour. This reaction results in the formation of dimethylsiloxy groups on the particle surface without significantly altering the particle diameter. The silanol content was determined by acid-base titration with sodium hydroxide and the relative content of silanol groups after surface modification was determined by dividing the silanol content of the modified silica by that of the unmodified silica (100% SiOH). The carbon content was determined by C,H,N analysis. In this work, a series of particles ranging from 14% SiOH (most hydrophobic) to 100% SiOH (most hydrophilic) were used.

### EXAMPLES

The invention will now be described by reference to the following examples, which are merely illustrative and are not to be construed as a limitation of the scope of the present invention. All temperatures are given in degrees centigrade; all solvents are highest available purity unless otherwise indicated.

### Methods—Preparation of Particle-Stabilised Emulsions

5 ml of oil, 5 ml of polar phase and the required mass of silica particles was emulsified in glass vessels (diameter 2.5 cm, length 7.5 cm) thermostatted at 25° C. The polar phase was either propane-1,2-diol or PEG300 and contained 4 mM NaCl to increase the conductivity. Emulsions were prepared using the powdered particle method. In this method, fumed silica particles were added as a powder on top of the most dense liquid phase (glycol) followed by the least dense phase (oil). Emulsification was achieved with an IKA Ultra-Turrax homogeniser fitted with a dispersing head of diameter 18 mm operating at 13,000 rpm for 5 minutes. This method removes the possibility that the initial location of the particles may influence the subsequent emulsion properties and so particles dictate the behaviour due solely to their inherent wettability. All emulsions prepared contained equal volumes of oil and polar phase and the effect of particle wettability (via % SiOH) was investigated. All emulsions contained 1 wt. % silica particles with respect to the sum of the mass of both liquid phases.

### Characterisation of Emulsions

The continuous phase of an emulsion was inferred by observing whether a drop of emulsion dispersed or remained when added to either the pure oil or pure polar phase used to prepare the emulsion. Glycol continuous emulsions disperse in glycol and remain as drops in oil, whereas oil continuous emulsions remain as drops in glycol but disperse in oil. A Jenway 3540 conductivity meter using Pt/Pt black electrodes was used to determine the conductivity of emulsions. Conductivity measurements were made immediately after emulsification. Low conductivity values were indicative of oil continuous emulsions whereas relatively high conductivity values were associated with glycol continuous emulsions doped with 4 mM NaCl. Emulsions were stored at room temperature (21±2° C.) in the vessels used during homogenisation. Photographs of the vessels were taken with a Panasonic DMC-FS15 digital camera.

### Transitional Inversions of Waterless Emulsions

### Paraffin Liquid/PEG300 Emulsions:

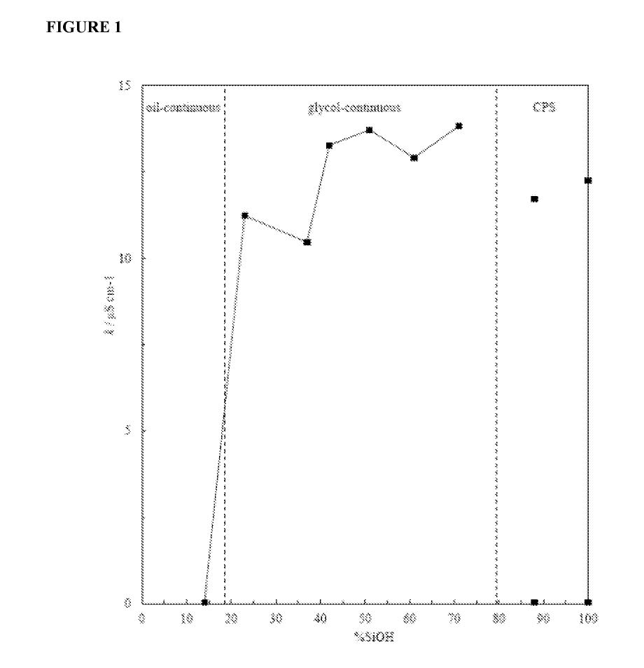

As demonstrated in FIG. 1 the conductivity and type of emulsions prepared from 50 vol. % paraffin liquid and 50 vol. % PEG300 containing 1 wt. % silica particles as a function of particle hydrophobicity is given. For complete phase separation (CPS) systems, conductivity fluctuates between that for oil and that for glycol containing 4 mM NaCl.

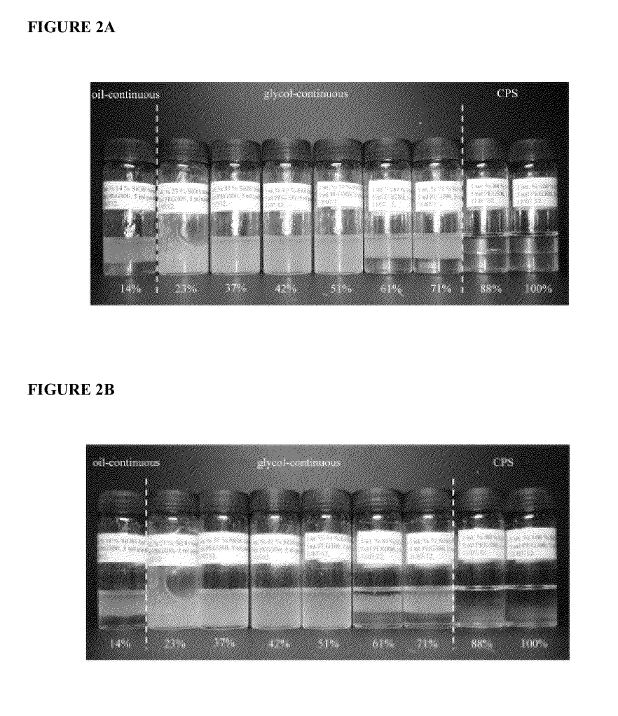

As demonstrated in FIGS. 2 A and B the appearance and type of emulsions prepared from 50 vol. % paraffin liquid and 50 vol. % PEG300 containing 1 wt. % silica particles as a function of particle hydrophobicity after 1 day (upper) and 1 week (lower) is given. The % SiOH on the silica is illustrated for each vial. In the absence of fumed silica particles, emulsions prepared from 50 vol % paraffin liquid, 50 vol % PEG300 and an identical methodology, completely separated into the individual phases within 60 seconds.

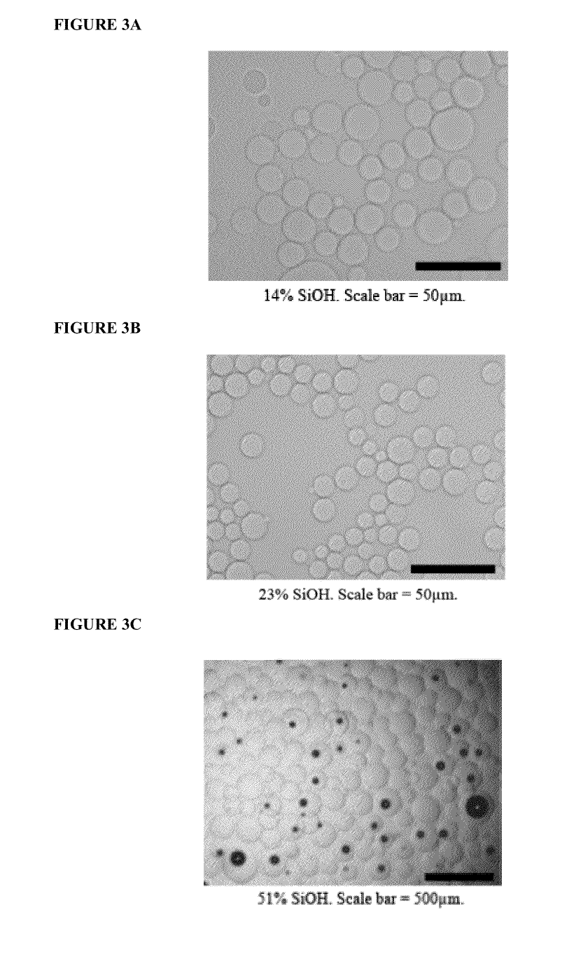

As demonstrated in FIGS. 3 A, B and C optical micrographs of dilute paraffin liquid/PEG 300 emulsions (φₒ=0.5) stabilised by 1 wt. % silica particles of varied wettability, viewed immediately after preparation are given. The % SiOH on the silica is shown for each figure, FIG. 3A=14% SiOH, with a scale bar of 50 μm; FIG. 3B=23% SiOH, with a scale bar of 50 μm; FIG. 3C=51% SiOH, with a scale bar of 50 μm.

The air bubbles which remain trapped within these emulsion formulations, as shown in FIG. 2, are also illustrated in optical microscopy. FIG. 3 illustrates that these air bubbles reside (are trapped) within the more viscous, dispersed paraffin liquid phase of emulsions stabilised by fumed silica nanoparticles with 51% surface silanol groups. This observation may be true for all opaque, white emulsions also but is highlighted more so within these translucent waterless systems.

### Additional Information Regarding Paraffin Liquid/PEG300 Emulsions:

### Emulsion Component Refractive Indices:

The refractive index of paraffin liquid and PEG300 where measured at 23° C. using an Abbe refractometer with water jacketed prisms. The observed difference in refractive indices corresponds to the translucent appearance of prepared emulsions as illustrated in FIG. 2.

| Sample | Refractive index (at 23° C.) |
|---|---|
| Paraffin liquid | 1.475 |
| PEG300 | 1.464 |

### Gelling of Individual Emulsion Components

FIG. 2 illustrates the thick, gel-like nature of the prepared paraffin liquid/PEG300 emulsions, especially of those stabilized by fumed silica particles with 23% surface silanol groups remaining. Inspection by optical microscopy (shown in FIG. 3) also highlights that for systems where gelling is significant (i.e. emulsions stabilized by 23% SiOH), the extent of dispersed droplet flocculation is greater, corresponding to the formation of structured, solid-like networks of the emulsion dispersed droplets. The dispersion of 2 wt % silica particles (equivalent to the maximum possible particle concentration in the prepared φₒ=0.5 emulsions) in each respective emulsion phase induces no visual increase in viscosity for PEG300 and a minor visual increase in viscosity for paraffin liquid. This observation is exaggerated dramatically when the particle concentration is increased to 5 wt % whereby PEG300 remains a free flowing liquid while paraffin liquid entirely gels.

Miglyol 812/propane-1,2-diol emulsions.

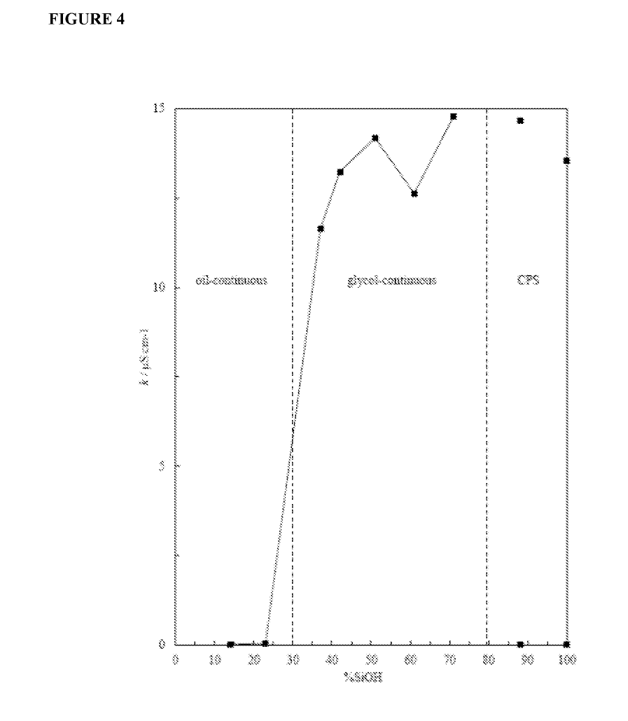

As demonstrated in FIG. 4 the conductivity and type of emulsions prepared from 50 vol. % miglyol 812 and 50 vol. % propane-1,2-diol containing 1 wt. % silica particles as a function of particle hydrophobicity is given. For complete phase separation (CPS) systems, conductivity fluctuates between that for oil and that for glycol containing 4 mM NaCl. Miglyol 812 is a fractionated coconut oil having a boiling range of 240-270° C. and composed of saturated C₈ (50-65%) and C₁₀ (30-45%) triglycerides.

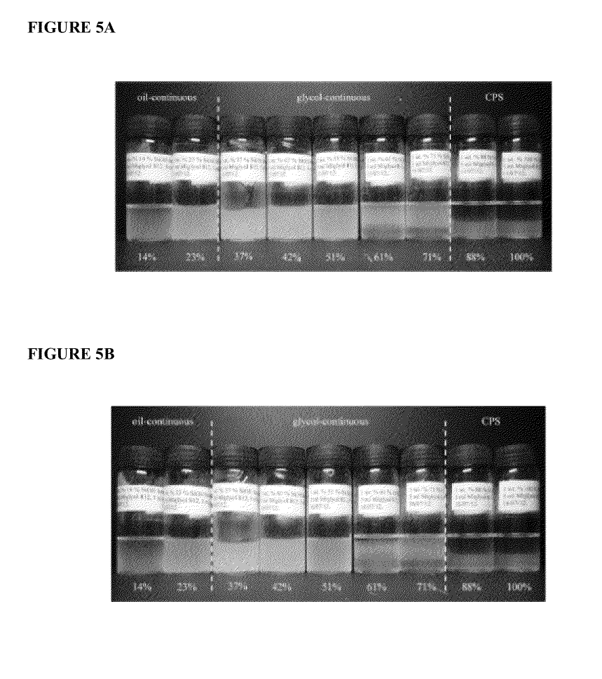

As demonstrated in FIGS. 5 A and B the appearance and type of emulsions prepared from 50 vol. % Miglyol 812 and 50 vol. % propane-1,2-diol containing 1 wt. % silica particles as a function of particle hydrophobicity after 1 day (upper) and 1 week (lower) is given. The % SiOH on the silica is illustrated. In the absence of fumed silica particles, emulsions prepared from 50 vol. % Miglyol 812, 50 vol. % propane-1,2-diol and an identical methodology, completely separated into the individual phases within 60 seconds.

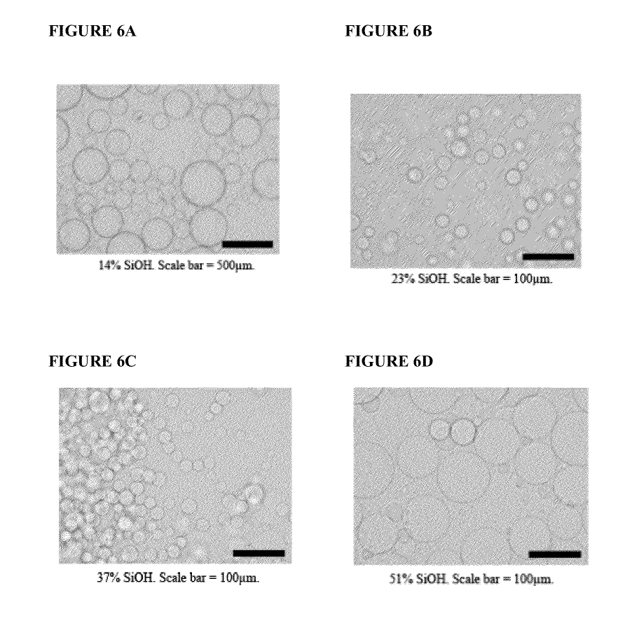

As demonstrated in FIGS. 6 A-D optical micrographs of dilute Miglyol 812/propane-1,2-diol emulsions (φₒ=0.5) stabilised by 1 wt. % silica particles of varied wettability, viewed immediately after preparation are given. The % SiOH on the silica in FIG. 6A is 14% SiOH, with a scale bar of 500 μm; FIG. 6B=23% SiOH, with a scale bar of 100 μm; FIG. 6C=37% SiOH, with a scale bar of 100 μm and in FIG. 6D=51% SiOH, with a scale bar of 100 μm.

The emulsions prepared from 50 vol. % Miglyol 812, 50 vol. % propane-1,2-diol and 1 wt. % fumed silica particles do not exhibit the significant gelling (see FIG. 5) as those shown for the previous PEG300/paraffin liquid systems (see FIG. 2). In accordance with this observation, we also do not view significant flocculation of the emulsions dispersed droplets during optical microscopy.

### Additional Information Regarding Miglyol 812/Propane-1,2-Diol Emulsions.

### Emulsion Component Refractive Indices:

The refractive index of Miglyol 812 and propane-1,2-diol were measured at 23° C. using an Abbe refractometer with water jacketed prisms. The observed difference in refractive indices corresponds to the translucent appearance of prepared emulsions as illustrated in FIG. 2.

| Sample | Refractive index (at 23° C.) |
|---|---|
| Miglyol 812 | 1.450 |
| Propane-1,2-diol | 1.432 |

### Determination of the Water Content in Emulsion Components by Karl Fischer Titration

The concentration of water present in components of the prepared emulsion systems was determined by Karl Fischer Titration. A selection of the emulsion components were analyzed and the determined water content is given subsequently.

| Sample | Water content/% |
|---|---|
| Paraffin liquid | 0.02 |
| PEG 300 | 1.03 |
| Propane-1,2-diol | 0.14 |

The above description fully discloses the invention including preferred embodiments thereof. Modifications and improvements of the embodiments specifically disclosed herein are within the scope of the following claims. Without further elaboration, it is believed that one skilled in the art can, using the preceding description, utilise the present invention to its fullest extent. Therefore, the examples herein are to be construed as merely illustrative and not a limitation of the scope of the present invention in any way. The embodiments of the invention in which an exclusive property or privilege is claimed are defined as follows.

1. A particle stabilized oil-in-polar (o/polar o/p) emulsion comprising:
a) a dispersed oil phase,
b) a continuous polar phase substantially free of water,
c) a particulate solid which is a silica particle dispersant possessing surface silanol groups (SiOH) sufficient to stabilize the emulsion, and where the emulsion is substantially free of emulsifiers, surfactants; and
wherein the oil phase (a) is dispersed as discontinuous droplets in the polar phase (b); the silica particle dispersant (c) is absorbed on the surface of the oil phase (a); and
the silica particle dispersant (c) is partially wetted by the polar phase (b).

2. A particle stabilized polar-in-oil emulsion which comprises:
a) a continuous oil phase,
b) a dispersed polar phase substantially free of water,
c) a particulate solid which is a silica particle dispersant possessing surface silanol groups (SiOH) sufficient to stabilize the emulsion, and where the emulsion is substantially free of emulsifiers, surfactants, and stabilizing polymers; and
wherein the polar phase (b) is dispersed as discontinuous droplets in the oil phase (a); the silica particle dispersant (c) is absorbed on the surface of the polar phase (b); and the silica particle dispersant is partially wetted by the oil phase (a).

3. The emulsion according to claim 1, wherein the emulsion further comprises an electrolytic component soluble in the polar phase.

4. The emulsion according to claim 2, wherein the emulsion further comprises an electrolytic component soluble in the polar phase.

5. The emulsion according to claim 1, wherein the oil phase and polar phase are of equal volume.

6. The emulsion according to claim 2, wherein the oil phase and polar phase are of equal volume.

7. The emulsion according to claim 1, wherein the oil phase and polar phase are in a 20:80 to 80:20 ratio to each other.

8. The emulsion according to claim 2, wherein the oil phase and polar phase are in a 20:80 to 80:20 ratio to each other.

9. The emulsion according to claim 1, wherein the oil phase comprises an oil, fat, or triglyceride, or a combination thereof.

10. The emulsion according to claim 2, wherein the oil phase comprises an oil, fat, or triglyceride, or a combination thereof.

11. The emulsion according to claim 9, wherein the oil phase comprises an oil selected from a group consisting of liquid paraffin oil, or a polyolefin, or a silicone oil, or a hydrocarbon oil, or a combination thereof.

12. The emulsion according to claim 10, wherein the oil phase comprises an oil selected from a group consisting of liquid paraffin oil, or a polyolefin, or a silicone oil, or a hydrocarbon oil, or a combination thereof.

13. The emulsion according to claim 9, wherein the oil phase comprises a fat, selected from a group consisting of at least one triglyceride.

14. The emulsion according to claim 10, wherein the oil phase comprises a fat, selected from a group consisting of at least one triglyceride.

15. The emulsion according to claim 1, wherein the polar solvent comprises a polyhydric alcohol substantially free of water

16. The emulsion according to claim 2, wherein the polar solvent comprises a polyhydric alcohol substantially free of water.

17. The emulsion according to claim 15, wherein the polar phase is selected from ethane-1,2-diol, propane-1,3-diol, propane-1,2-diol, butane-1,4-diol, butane-1,3-diol, butane-1,2-diol, or polyethylene glycol substantially free of water.

18. The emulsion according to claim 16, wherein the polar phase is selected from ethane-1,2-diol, propane-1,3-diol, propane-1,2-diol, butane-1,4-diol, butane-1,3-diol, butane-1,2-diol, or polyethylene glycol substantially free of water.

19. The emulsion according to claim 1, wherein the polar phase is selected from, ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether.

20. The emulsion according to claim 2, wherein the polar phase is selected from, ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether.

21. The emulsion according to claim 1, wherein the polar phase is propane-1,2-diol present in an amount of about 1% to about 50% of total volume of (a) and (b) phases.

22. The emulsion according to claim 2, wherein the polar phase is propane-1,2-diol present in an amount of about 1% to about 50% of total volume of (a) and (b) phases.

23. The emulsion according to claim 1, wherein the silica primary particle dispersant has a mean diameter less than 60 nm.

24. The emulsion according to claim 2, wherein the silica primary particle dispersant has a mean diameter less than 60 nm.

25. The emulsion according to claim 1, wherein the stable aggregate of the silica primary particle dispersants is about 100 to about 500 nm in diameter.

26. The emulsion according to claim 2, wherein the stable aggregate of the silica primary particle dispersants is about 100 to about 500 nm in diameter.

27. The emulsion according to claim 1, wherein the residual density of surface silanol groups is about 14% to about 100% of the silica particle's surface area (as defined by original silanol content).

28. The emulsion according to claim 2, wherein the residual density of surface silanol groups is about 14% to about 100% of the silica particle's surface area (as defined by original silanol content).

29. The emulsion according to claim 1, wherein the silica dispersant (c) has a calculated contact angle θ of θ<90° for o/w emulsions.

30. The emulsion according to claim 2, wherein the silica dispersant (c) has a calculated contact angle θ of θ>90° for w/o emulsions.

31. The emulsion according to claim 1, wherein the silica particle dispersants are present in the amount of about 0.1 to 10% weight %.

32. The emulsion according to claim 2, wherein the silica particle dispersants are present in the amount of about 0.1 to 10% weight %.

33. The emulsion according to claim 1 further comprising at least one pharmaceutically or cosmetically acceptable active agent.

34. The emulsion according to claim 2 further comprising at least one pharmaceutically or cosmetically acceptable active agent.

35. The emulsion according to claim 33 further comprising at least one additive agent selected from a lipophilic gelling agent, preservative, a perfume, filler, or colorant.

36. The emulsion according to claim 34 further comprising at least one additive agent selected from a lipophilic gelling agent, preservative, a perfume, filler, or colorant.

37. The emulsion according to claim 33, wherein the emulsion is used in a cosmetic or pharmaceutical preparation.

38. A process for preparation of an oil-in-polar phase (o/p) emulsion comprising:
a) a dispersed oil phase;
b) a continuous polar phase substantially free of water;
c) a silica particle dispersant possessing surface silanol groups (SiOH) sufficient to stabilize the emulsion, and where the emulsion is substantially free of emulsifiers, surfactants, and stabilizing polymers;
d) adding the silica particle dispersant (c) as a powder on top of the most dense liquid phase of (a) or (b);
e) adding the least dense phase of (a) or (b) on top of (d); and
f) mixing or homogenizing (e) to produce an emulsion; and
wherein the oil phase (a) is dispersed as discontinuous droplets in the polar phase (b);
the silica particle dispersant (c) is absorbed on the surface of the oil phase (a); and
the silica particle dispersant is partially wetted by the polar phase (b); and wherein the oil or polar phase may optionally comprise at least one pharmaceutically or cosmetically acceptable active agent.

39. A process for preparation of a product polar phase-in-oil (p/o) emulsion comprising:
a) a continuous oil phase;
b) a dispersed polar phase substantially free of water;
c) a silica particle dispersant possessing surface silanol groups (SiOH) sufficient to stabilize the emulsion, and where the emulsion is substantially free of emulsifiers, surfactants, and stabilizing polymers;
d) adding the silica particle dispersant (c) as a powder on top of the most dense liquid phase of (a) or (b);
e) adding the least dense phase of (a) or (b) on top of (d); and
f) mixing or homogenizing (e) to produce an emulsion; and
wherein the polar phase (b) is dispersed as discontinuous droplets in the oil phase (a); the silica particle dispersant (c) is absorbed on the surface of the polar phase (b); and the silica particle dispersant is partially wetted by the oil phase (a) and wherein the oil or polar phase may optionally comprise at least one pharmaceutically or cosmetically acceptable active agent.

40. A product emulsion stable to creaming and coalescence, prepared by the process according to claim 38.

41. A product emulsion stable to creaming and coalescence, prepared by the process according to claim 39.

42. The product according to claim 33 wherein the at least one pharmaceutically or cosmetically acceptable active agent is a UV-A filter substance, a UV-B filter substance, a deodorant or antiperspirant, an antioxidant, an insect repellent, a vitamin, or an antimicrobial agent.

43. The product according to claim 34 wherein the at least one pharmaceutically or cosmetically acceptable active agent is a UV-A filter substance, a UV-B filter substance, a deodorant or antiperspirant, an antioxidant, an insect repellent, a vitamin, or an antimicrobial agent.
